(19) United States
(12) Patent Application Publication
(10) Pub. No.: US 2004/0049147 A1
(43) Pub. Date: Mar. 11, 2004

(54) Cold-contact electrode system for iontophoresis

(76) Inventors: Susann Edel, Grunwald (DE); Rosemarie Winkler, Penzberg (DE)

Correspondence Address: SQUIRE, SANDERS & DEMPSEY L.L.P, 600 HANSEN WAY, PALO ALTO, CA, 94304-1043, US

(21) Appl. No.: 10/465,990

(22) PCT Filed: Aug. 19, 2002
(86) PCT No.: PCT/EP02/09251

(30) Foreign Application Priority Data
Aug. 23, 2001 (DE) 101 41 254.1

(51) Int. Cl. A61N 1/30

(52) U.S. Cl. 604/20

(57) ABSTRACT

A portable iontophoresis cold-contact electrode system having an outwardly raised, arched, electrically conducting electrode surface area (13) that can be cooled by a built-in sensor-regulated cooling element (30) in a defined way for the simultaneous cooling of the epidermis and the iontophoretic introduction of medicinal and cosmetic active substances through the epidermis into the underlying body tissue. In this process, the cooling of the epidermis makes possible an abrasion of horny skin cells on the skin surface, and also a conditioning and a better conductivity of the skin in order to funnel medicinal or cosmetic iontophoretic active substances more rapidly and more reliably through the human skin membrane into the underlying body tissue. The same electrode surface area can, if necessary, also be used to heat the skin by switching at the iontophoresis control unit if the individual conditions of the skin to be treated require it. Furthermore, the iontophoresis cold-contact electrode system comprises an electrode cover (12) that is provided with a central large opening and that firmly clamps an active-substance pad applied to the electrode surface area (13) over the entire area when screwed onto the electrode body (11). The iontophoresis cold-contact electrode system likewise comprises an active-substance pad (20) that is composed of a flexible, porous, electrically nonconducting material preferably roughened at the surface and that is impregnated with the iontophoretic active substance.

### DESCRIPTION

The present invention relates to an iontophoresis cold-contact electrode system having a defined, cooled electrode surface area for the simultaneous process of iontophoresis pretreatment by abrading the cooled, dry, horny skin layers with poor current conduction and the optimized iontophoresis treatment due to the improved penetration of the iontophoresis current into the cooled skin membrane having better current conduction for iontophoretically introducing cosmetic or medicinal active substances via the human skin membrane into the underlying body tissue.

Iontophoresis, which has been used for many years, is a method of introducing electrically charged, that is to say ionized, cosmetic or medicinal active substances via the epidermis into the underlying body tissue. In this process, the electrically charged active-substance mixture is applied either directly, or indirectly in an active-substance carrier, to the user electrode, termed the contact electrode, which then makes contact with the skin at the desired point, in which process only the active-substance mixture or the active-substance carrier, but not the electrode itself, is allowed to come into contact with the skin. The counterelectrode, termed the ground electrode, is connected to another skin area. An electric voltage is now applied between the two electrodes and generates an electric field, with the result that a so-called iontophoresis current flows through the body and transports the active-substance molecular ions through the skin. The electric charge of the active substance determines, under these circumstances, the polarity at the two electrodes. For example, in the case of a positive active-substance charge, the contact electrode is polarized positively and the ground electrode negatively, with the result that the positively charged active-substance molecules are transported away from the contact electrode in the direction of the ground electrode through the epidermis into the underlying tissue.

As already mentioned in Patent Publication DE 694 25 728 T2, the electrical conductivity of the skin can be modified by applying ice packs or hot packs. The application of the cold packs or hot packs would, however, only be preconditioning or postconditioning, respectively, with short-term effect on the skin and with the additional disadvantage that this existing technique does not make possible a rapid switching to heating or cooling and a lasting influence on the skin surface temperature immediately the treatment has finished.

For many years, it has also been known that an improvement in the iontophoresis or the penetration behaviour of certain active substances into the skin is achieved by severely precooling the skin. On the one hand, this is due to the fact that the dry, horny skin layers with poor electrical conductivity can be abraded better after cooling and, on the other hand, it is due to the fact that the iontophoresis current penetrates better into the cooled and, consequently, more electrically conducting skin membrane. The precooling of the skin has hitherto been achieved by ice packs or other cold packs that are applied to the respective skin area prior to the actual iontophoresis treatment. The disadvantage of this method is that a plurality of processes has to be performed and that the precooling of the skin rapidly eases off and, consequently, the achievable cooling effect barely still persists during the iontophoresis and an abrasion effect can also not be achieved.

The object of the present invention is to provide an iontophoresis electrode system that eliminates the disadvantages described above and makes possible improved iontophoresis. This object is achieved, according to the invention, by an iontophoresis cold-contact electrode system having the features of claim 1. The subclaims each define advantageous and preferred embodiments of the invention.

Not known hitherto was an iontophoresis electrode system that combines simultaneous iontophoresis and skin cooling. The present invention provides a portable iontophoresis cold-contact electrode that makes possible iontophoresis with simple means and simultaneously cools the skin. In particular, this process ensures the easy replacement of the active-substance carrier provided with an abrasive surface, termed the active-substance pad, the firm fixing of the active-substance pad on the electrode surface, the very good facility for removing active-substance residues from the electrode after use and totally problem-free handling during use. In addition, the present iontophoresis cold-contact electrode is suitable, in particular, in cosmetic use as the result of the special electrode shape for the various difficult skin regions in the face and, above all, it is easy for the user to use during treatment.

The iontophoresis cold-contact electrode system according to the invention comprises an electrode body having an internally cooled electrically conducting electrode surface raised and arched outwards in such a way that even the narrow face areas around the nose, mouth, eyes and ears can readily be reached by said raised, arched shape. If necessary, the cooling element in the interior of the electrode body can also be used as a heating element for reheating the skin to normal body temperature by switching at the control unit. This cold/hot effect is additionally used for skin conditioning.

Furthermore, the iontophoresis cold-contact electrode system according to the invention comprises an electrode cover having a central large opening that is such that, when screwed onto the electrode body, the electrode cover with its cover flange firmly clamps, over the entire surface, the active-substance pad previously applied to the electrode surface and projecting somewhat onto the electrode surface in such a way that the active-substance pad projects in a firmly fixed way out of the cover opening.

The iontophoresis cold-contact electrode system according to the invention likewise comprises an active-substance pad made of a flexible, porous, electrically nonconducting material that is suitably severely roughened at the surface and that is impregnated like a fully soaked sponge with the electrically charged cosmetic or medicinal active-substance mixture. Advantageously, the active-substance pad is stored in an airtight package as a disposable active-substance pad and is applied to the electrode surface only immediately before use and is fixed for the treatment as described above. In this connection, the amount of active substance contained in a disposable active-substance pad is precisely adjusted for one treatment, with the result that the spent pad can be disposed of as waste at the end of the treatment and the electrode can be cleaned for a subsequent, fresh treatment.

EXPLANATION OF THE FIGURES

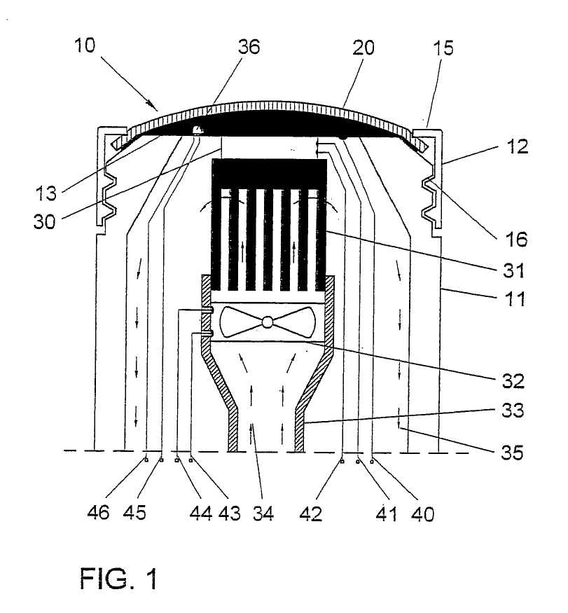

FIG. 1 shows, by way of example, a cross-sectional drawing of an iontophoresis cold-contact electrode system (10) according to the invention, comprising an electrode body (11) having an upwardly arched, electrically conducting electrode surface area (13) and the applied active-substance pad (20), which is firmly clamped two-dimensionally on the upwardly arched electrode surface area (13) by the cover flange (15) of the upwardly open electrode cover (12) and projects in a raised manner beyond the electrode cover (12). The firm clamping of the active-substance pad (20) by the cover flange (15) is achieved by firmly screwing the electrode cover (12) onto the external thread of the electrode body (11) by means of its internal thread (16). The rear termination of the electrode body is shown symbolically as a broken line.

The electrode surface area (13) is cooled or heated in a defined way by means of a cooling/heating element, preferably a commercial Peltier element (30), mounted at its inner side if this is advantageous for conditioning the skin better for the introduction of the iontophoretic active substances.

In the cooling mode, the Peltier element (30) at the side of the electrode cools down and thus absorbs the heat from the electrode surface area (13) and delivers it at its other side to a cooling body (31) that is fitted there and whose cooling ribs are cooled by an air cooling system. In said air cooling system, the fan (32) fitted in front of the cooling body (31) takes in the cool outside air (34) through the air guiding scoop (33) and blows the heated used air (35) through the cooling ribs of the cooling body (31) and through the interior of the electrode body (11) to the outside.

In the heating mode, the current direction in the Peltier element (30) is reversed in polarity and, under these conditions, the Peltier element at the side of the electrode heats up and thus heats the electrode surface area (13). Under these conditions, the air cooling system can additionally be used for temperature regulation.

The temperature of the electrode surface area (13) and, consequently, also that of the clamped-on active-substance pad (20) is determined by the temperature sensor (36) mounted in the electrode surface (13) and is fed as actual temperature value to a temperature regulator in the iontophoresis control unit, which regulates the desired cooling or heating temperature precisely. The conductors (40)-(46) are the conductors connecting the electrical components of the cold-contact electrode system to the iontophoresis control unit, conductor (40) being connected to the electrode surface area (13) and carrying the iontophoresis current, the conductors (41-42) conducting the current to the Peltier element (30), the conductors (43-44) supplying the ventilator (32) and the conductors (45-46) making the connection to the temperature sensor (36).

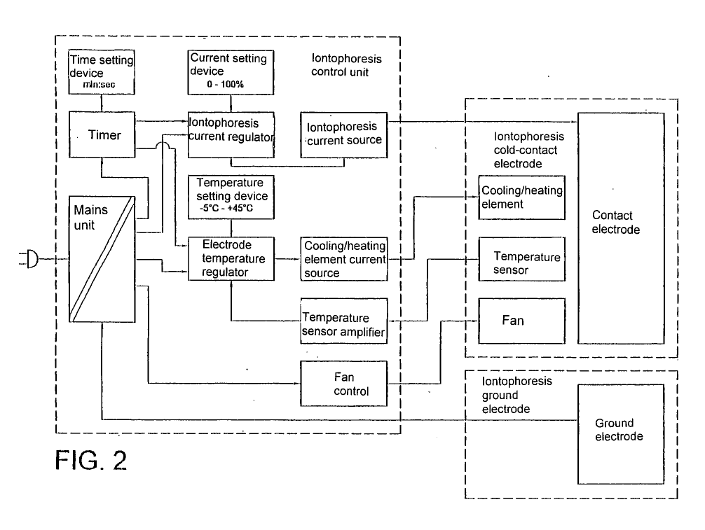

FIG. 2 shows, by way of example, a construction of an iontophoresis unit as a block circuit diagram, which unit has an iontophoresis cold-contact electrode system according to the invention, the iontophoresis ground electrode and the iontophoresis control unit. The iontophoresis current regulator regulates precisely the intensity and the variation of the iontophoresis current with time by means of the current setting device and the time setting device, respectively, and, driven by the iontophoresis power source, the current flows into the surface of the iontophoresis cold-contact electrode, then through the clamped-on iontophoresis active-substance pad into the patient's skin area, then through the patient's body to the ground electrode connected to another body point and then back to the mains unit in the control unit.

Cooling or heating of the surface area of the iontophoresis cold-contact electrode is made possible by a cooling-and-heating element that is connected to a temperature-regulating circuit, comprising a temperature setting device, electrode temperature regulator, reversible power source and a temperature sensor mounted on the contact electrode. The fan necessary for the air cooling of the cooling body is controlled by the fan control in the control unit.

To cool/heat the electrode surface area (13), a cooled/heated and preferably fluid medium, in particular a gas, can be used that is conveyed past it and is provided by a suitable guidance system.

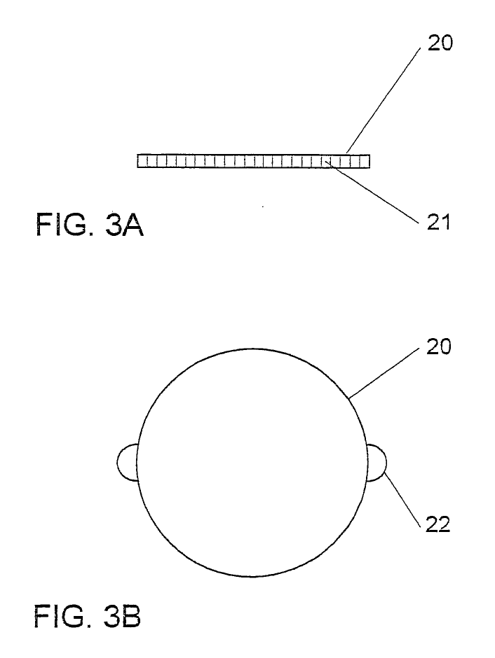

FIG. 3A shows, by way of example, as a cross-sectional drawing, a disposable active-substance pad (20) impregnated with the iontophoretic active substance (21) and made of an absorbent, porous material suitably severely roughened at the surface for the desired skin abrasion. The disposable active-substance pad (20) should be packed airtightly in a single blister for better durability and simplicity of use, and disposed of as a disposable pad after a treatment.

FIG. 3B shows by way of example a disposable active-substance pad (20) with additional attachments (22) for easier removal from the packaging.

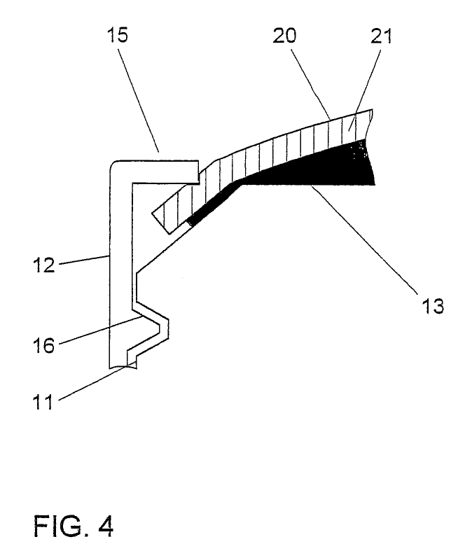

FIG. 4 shows in a cross-sectional drawing enlarged as a detail from FIG. 1 how the firmly screwed electrode cover (12) with its cover flange (15) clamps the active-substance pad (20), impregnated with the iontophoretic active substance, at the edge and firmly clamps it onto the upwardly arched, electrically conducting electrode surface (13) over the entire surface, and how the active-substance pad (20) projects beyond the electrode cover (12), with the result that, during the iontophoresis treatment, only the active-substance pad (20) touches the skin area of the patient to be treated for the purpose of transmitting the active substance.

1. Iontophoresis cold-contact electrode system having a defined cooled electrode surface (13) for the simultaneous use of the cooling and of the iontophoresis on human skin for the purpose of abrasion of horny cells of the upper skin layer and the simultaneous introduction of iontophoretic active substances into the human skin.

2. Iontophoresis cold-contact electrode system according to claim 1, characterized in that the cooled temperature of the electrode surface (13) is determined by an integrated temperature sensor (36) and is controlled by regulation to a temperature of down to −5 (minus five) degrees Celsius in a defined and selectable way.

3. Iontophoresis cold-contact electrode system according to claim 2, characterized in that the coolable electrode surface (13) is cooled in a defined way by a Peltier element (30).

4. Iontophoresis cold-contact electrode system according to claim 2, characterized in that the coolable electrode surface (13) is connected to a guidance system and it is cooled by conveying a suitably cooled, fluid coolant past it.

5. Iontophoresis cold-contact electrode system according to claim 4, characterized in that the coolant is a gas.

6. Iontophoresis cold-contact electrode system according to any one of the preceding claims, characterized in that the same electrode surface (13) that is then applied to the human skin can, if necessary, also be heated in a defined way preferably to +45 degrees Celsius and thus also heats the human skin again if there is a corresponding need.

7. Iontophoresis cold-contact electrode system according to claims 3 and 6, characterized in that the heating of the electrode surface (13) takes place by switching the polarity at the Peltier element (30).

8. Iontophoresis cold-contact electrode system according to claim 6, characterized in that the electrode surface (13) is connected to a guidance system and it is heated by conveying a suitably heated medium past it.

9. Iontophoresis cold-contact electrode system according to claim 8, characterized in that the medium used to heat the electrode surface (13) is a gas.

10. Iontophoresis cold-contact electrode system according to any one of the preceding claims, characterized in that it is equipped with a device for clamping to an electrode body (11) which serves to fix active-substance pads (20) impregnated with iontophoretic active substances rapidly and simply.

11. Iontophoresis cold-contact electrode system according to claim 10, characterized in that the clamping device is constructed in such a way that the active-substance pad (20) composed of a porous, nonconducting material and impregnated with the iontophoretic active substance is firmly clamped onto the electrode body (11) having an outwardly arched, electrically conducting electrode surface (13) by a cover flange (15) of an upwardly open electrode cover (12) over the entire area onto the electrode surface (13) in such a way that the active-substance pad is fixed so as to project in a raised manner beyond the electrode body and can thereby be conveyed over the human skin and closes an iontophoresis current circuit to a ground electrode connected to another body region.

12. Iontophoresis cold-contact electrode system according to claim 11, characterized in that the clamping device is constructed in such a way that the electrode cover (12) is firmly screwed onto an external thread of the electrode body (11) by means of an internal thread (16).

13. Iontophoresis cold-contact electrode system according to claim 11, characterized in that the clamping device is constructed in such a way that the electrode cover (12) is fixed by a bayonet closure to the electrode body (11).

14. Iontophoresis cold-contact electrode system according to claim 11, characterized in that the clamping device is constructed in such a way that the electrode cover (12) is fixed to the electrode body (11) by a latching cam.

15. Iontophoresis cold-contact electrode system according to claim 11, characterized in that the clamping device is constructed in such a way that the electrode cover (12) is pressed onto and thereby fixed to the electrode body (11) by a press seating.

16. Iontophoresis cold-contact electrode system according to any one of claims 10-15, characterized in that the active-substance pad (20) is composed of a flexible, porous nonconducting material roughened on the surface area and is impregnated with one or more electrically conducting iontophoretic active substances dissolved in liquid or gel or cream.

17. Iontophoresis cold-contact electrode system according to claims 10-16, characterized in that the active-substance pad (20) is impregnated with medicinally active substances that are intended for introduction into the human skin.

18. Iontophoresis cold-contact electrode system according to any one of claims 10-17, characterized in that the active-substance pad (20) is impregnated with cosmetic active substances that are intended for introduction into the human skin.

19. Iontophoresis cold-contact electrode system according to any one of claims 10-18, characterized in that the active-substance pad (20) is impregnated with nutrition-supplementing active substances that are intended for introduction into the human skin.

20. Iontophoresis cold-contact electrode system according to any one of claims 10-19, characterized in that the active-substance pad (20) is constructed in circular shape.

21. Iontophoresis cold-contact electrode system according to any one of claims 10-19, characterized in that the active-substance pad (20) is constructed as a polygon.

22. Iontophoresis cold-contact electrode system according to any one of claims 10-21, characterized in that the active-substance pad (20) has laterally fitted gripping extensions (22) that serve for better removal.

23. Iontophoresis cold-contact electrode system according to any one of claims 10-22, characterized in that the active-substance pad (20) is a disposable active-substance pad.
